(19) United States
(12) Patent Application Publication
(10) Pub. No.: US 2016/0106326 A1
(43) Pub. Date: Apr. 21, 2016

(54) Pressure Wave Measurement of Blood Flow

(71) Applicant: Google Inc., Mountain View, CA (US)

(72) Inventors: Vikram Singh Bajaj, Mountain View, CA (US); Andrew Homyk, Belmont, CA (US)

(21) Appl. No.: 14/519,765

(22) Filed: Oct. 21, 2014

(51) Int. Cl. A61B 5/0285; A61B 5/00; A61B 8/06

(52) U.S. Cl. CPC A61B 5/0285; A61B 8/06; A61B 5/7278

(57) ABSTRACT

Methods and devices for measuring blood flow velocity are provided. The device may include a wave source and at least two detectors positioned along a blood vessel. The wave source, which may include an ultrasound transducer or a mechanical source, is configured to induce a pressure wave in blood flowing in a blood vessel. In one example, the detectors are both positioned downstream of the wave source, with respect to the direction of blood flow. In another example, one detector is positioned upstream of the wave source, and a second detector is positioned downstream of the wave source. The difference in time it takes for the induced pressure wave to reach the first and the second detectors is indicative of the velocity of blood flow in the vessel.

### BACKGROUND

Unless otherwise indicated herein, the materials described in this section are not prior art to the claims in this application and are not admitted to be prior art by inclusion in this section.

A number of scientific methods have been developed in the medical field to evaluate a person's health state. A person's health state may, for example, be evaluated based on the measurement of one or more physiological parameters, such as blood pressure, pulse rate, skin temperature, or galvanic skin response (GSR). In a typical scenario, these measurements may be taken in the home or a health-care setting by using several discrete devices or sensors and, in some cases, by drawing blood or other bodily fluid. For most people, the measurements or blood tests are performed infrequently, and changes in a physiological parameter, which may be relevant to health state, may not be identified, if at all, until the next measurement is performed.

Further, some methods for detection and characterization of physiological signals often suffer from a low signal-to-noise ratio (SNR), since the signal of interest is typically weak in comparison to the background. This can make discerning between the signal of interest and signals produced by other analytes, particles, and tissues present in the blood and elsewhere in the body very difficult, especially where the measurements are taken non-invasively from outside the body.

In one example, changes in blood pressure can be non-invasively detected by measuring pulse transit time. For each heartbeat, the heart ejects a stroke volume to the arteries. The time it takes the propagating pressure wave caused by the heartbeat to arrive in a peripheral arterial site is called pulse transit time (PTT). As the propagating pressure wave results in a local expansion of the artery, the PTT is related to arterial wall stiffness and blood pressure, which can be indicators of cardiac health. However, PTT does not measure the flow rate of the blood, but rather the propagation velocity of the pressure wave. Moreover, the measurement frequency of PTT is limited by the time of each heart cycle, which can limit detection sensitivity and resolution.

### SUMMARY

A device may be provided for measuring blood flow velocity by generating a pressure wave at a point along an artery and detecting the wave at two detectors, separated by a distance along the artery. The difference in time it takes for the pressure wave to arrive at the first and second detectors is indicative of the flow rate of the blood. Blood flow may be detected as a difference in velocity of the waves detected at each detector, a difference in phase, or a difference in frequency (Doppler effect). The device may also include a wave source, which may include an ultrasound transducer, an electromagnetic source, or a mechanical source.

Some embodiments of the present disclosure provide a device including: a wave source configured to induce a pressure wave in blood flowing in a blood vessel; a first detector positioned along the vessel and configured to detect a pressure wave in the blood flowing in the blood vessel; and a second detector positioned along the vessel and configured to detect a pressure wave in the blood flowing in the blood vessel, wherein the second detector is positioned downstream of the first detector with respect to a direction of blood flow in the blood vessel.

Further embodiments of the present disclosure provide a method including: (1) inducing a pressure wave in blood flowing in a blood vessel; (2) detecting the induced pressure wave at a first detector positioned along the blood vessel; (3) detecting the induced pressure wave at a second detector positioned along the blood vessel, wherein the second detector is positioned downstream of the first detector with respect to a direction of blood flow in the blood vessel; (4) determining a first time at which the pressure wave is detected by the first detector and a second time at which the pressure wave is detected by the second detector; and (5) calculating a time difference between the first time and the second time.

Still further embodiments of the present disclosure provide a method including: (1) inducing a pressure wave in blood flowing in a blood vessel; (2) detecting the induced pressure wave at a first detector positioned along the blood vessel; (3) detecting the induced pressure wave at a second detector positioned along the blood vessel, wherein the second detector is positioned downstream of the first detector with respect to a direction of blood flow in the blood vessel; (4) determining a first characteristic of the pressure wave detected by the first detector and a second characteristic of the pressure wave detected by the second detector; and (5) determining a difference between the first characteristic and the second characteristic, wherein the difference is indicative of a velocity of the blood flowing in the blood vessel.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates an example of a device for measuring blood flow velocity.

FIG. 2 is a schematic illustration of the example device of FIG. 1.

FIG. 3 illustrates another example of a device for measuring blood flow velocity.

FIG. 4 is a flow chart of an example method, according to an example embodiment.

FIG. 5 is a flow chart of an example method, according to an example embodiment.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

I. Overview

A device may measure blood flow by generating a pressure wave at a point along a blood vessel and detecting the wave at two detectors, separated by a distance along the vessel. The wave source may be provided as any device or mechanism capable of inducing a pressure wave in the blood vessel. For example, the wave source may be an ultrasound transducer, an electromagnetic source, or a mechanical source. In one example, the wave source is provided between the two detectors. In another example, the wave source is positioned upstream of both of the two detectors. The time at which the induced pressure wave arrives at the first and second detectors is determined and the difference provides an indication of the blood flow velocity.

Information regarding blood pressure may be inferred from the blood flow velocity detected by the device. Relative changes in blood pressure may be determined directly from blood flow measurements. With additional calibration, an absolute blood pressure can also be determined from blood flow. Changes in blood flow velocity at a point along a vessel over time may also be indicative of a medical condition. Differences in blood flow at various points along a vein or other vessel can also be used to identify blockages in the vasculature. Properties, such as elasticity, of vessel walls may also be inferred from the transit time of the pressure wave, which can be indicative of vascular health.

The disclosed technique can generate a pressure wave in the vessel at a higher frequency than pressure waves are generated by the beating of the heart. Accordingly, this technique can provide better temporal resolution of the detected signal. Further, the detected signal can be more robust in the presence of noise. In some examples, the induced pressure waves may be modulated to further distinguish them from any naturally-occurring biological waves. For example the induced pressure wave may be modulated in a detectable pattern that may be distinguished from the background.

It should be understood that the above embodiments, and other embodiments described herein, are provided for explanatory purposes, and are not intended to be limiting.

II. Example Devices for Measuring Blood Velocity

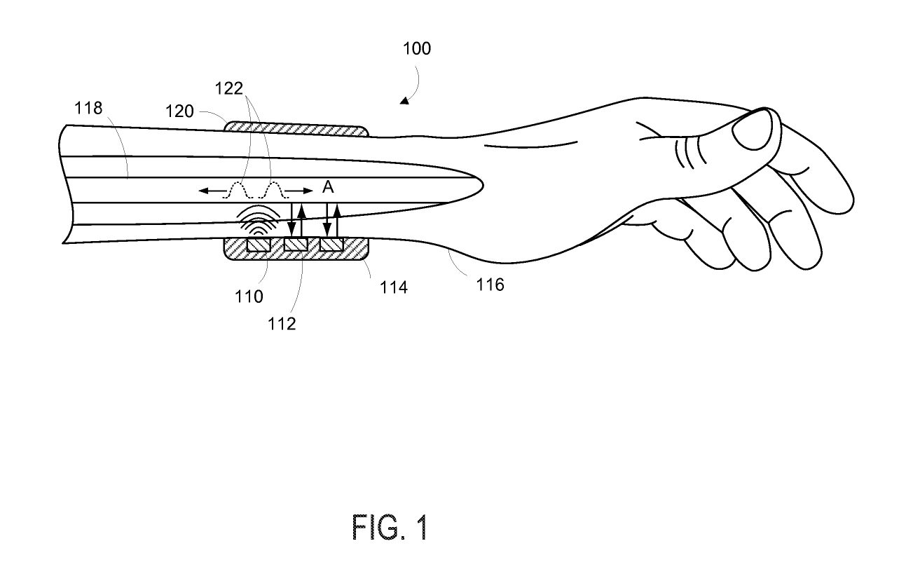

As shown in FIG. 1, a device 100 for measuring blood velocity may include a wave source 110 and at least a first detector 112 and a second detector 114. Detectors 112, 114 may each be provided as a detector array, such as an ultrasonic phased array, or a CMOS or CCD imaging detector. Alternatively, the device 100 may, in some embodiments, include a single continuous detector element capable of detecting pressure, and a position of maximum pressure, along its length. In some examples, the device 100 may comprise or form a part of a body-mountable device. In the embodiment illustrated in FIG. 1, the device 100 is placed on a human wrist 116 proximate to a blood vessel 118. The device 100 may be held against the body with a mount 120, such as a strap.

The wave source 110 may be any mechanism configured to induce a pressure wave 122 in blood flowing in the blood vessel 118. For example, the wave source may be an ultrasound transducer for producing an acoustic wave. Alternatively, the wave source 110 may be provided as an electromagnetic source capable of generating a photoacoustic wave caused by the photothermal expansion of light absorbing elements in the blood. The electromagnetic source may be an optical source in the visible range (˜520 nm) or infrared range, where light absorption in the blood is high as compared to surrounding tissues. The wave source may also include a mechanical means for inducing a pressure wave. For example, the source may include a mechanical element for periodically tapping the surface of the skin in an area proximal to a blood vessel. The induced pressure wave 122 will travel upstream and downstream away from the source, with respect to the direction of blood flow A.

Further, in some examples, the wave source 110 may be adapted to modulate one or more characteristics the induced pressure wave to distinguish it from the background. For example, the frequency, phase and/or intensity of the pressure wave 122 may be modulated.

In the embodiment shown in FIG. 1, the first detector 112 and the second detector 114 are both positioned downstream of the wave source 110, with the first detector 112 being upstream of the second detector 114. The detectors 112, 114 may be provided as any detector capable of detecting a pressure wave in the blood flowing in the blood vessel 118. For example, the detectors may be piezoelectric sensors, such as ultrasound transducers. In principle, the wave source 110 periodically generates a pressure wave in the vessel, which may result in an expansion of the arterial walls or motion of red blood cells in the blood. The detectors 112, 114 may detect the local change in blood volume, blood motion, or the propagating pressure wave itself. Laser speckle contrast imaging, optical absorption, magnetism, electrical or acoustical impedance, magnetic resonance, are some examples of detection methods that may be employed.

The wave source 110 and each of the detectors 112, 114 may respectively be spaced apart on the order of tens of millimeters. For example, the wave source 110 and each of the detectors may be spaced between about 50 and 100 millimeters apart. In general, the spacing between each of the detectors and the wave source is selected to provide enough time delay between the signals detected at the first 112 and second 114 detectors to distinguish a difference. On the other hand, if the detectors are placed too far away from the wave source, then the resulting pressure wave may be too weak to be distinguished over the background noise.

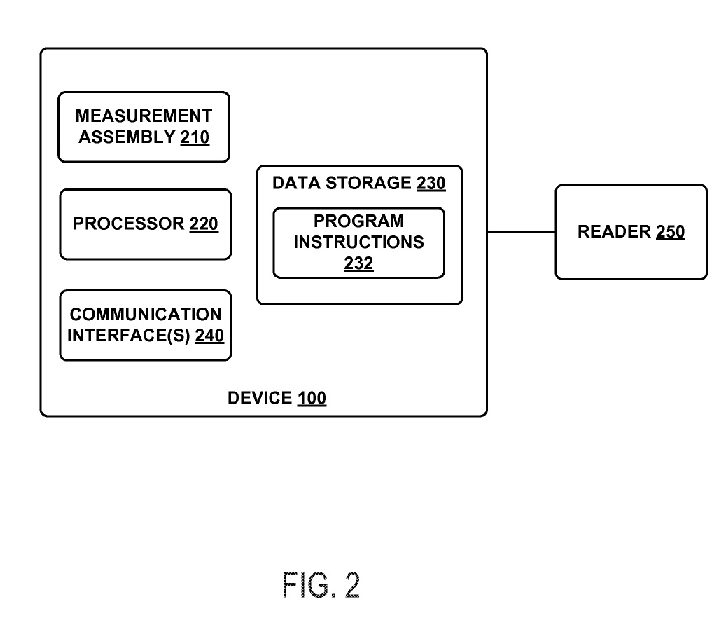

A schematic diagram of device 100 is illustrated in FIG. 2. In addition to a measurement assembly 210, which may include the wave source 110 and the detectors 112, 114, the device 100 may include a processor 220, data storage 230, and a communication interface 240 for communicating collected data to a reader 250. The communication interface 240 may include any means for the transfer of data, including both wired and wireless communications, such as a universal serial bus (USB) interface, a secure digital (SD) card interface, a plain old telephone service (POTS) network, a cellular network, a fiber network and a data network. In one embodiment, the communication interface 240 includes a wireless transceiver for sending and receiving communications to and from the server. The reader 250 may be any remote computing device such as a remote server, smart phone, digital assistant, or other portable computing device. The device 100 and reader 250 may also be configured to communicate with one another via any communication means. Data storage 230 is a non-transitory computer-readable medium that can include, without limitation, magnetic disks, optical disks, organic memory, and/or any other volatile (e.g. RAM) or non-volatile (e.g. ROM) storage system readable by the processor 220. The data storage 230 can store indications of data, such as sensor readings, program settings (e.g., to adjust behavior of the device 100), user inputs (e.g., from a user interface on the device 100 or communicated from a remote device), etc. The data storage 230 can also include program instructions 232 for execution by the processor 220 to cause the device 100 to perform processes specified by the instructions. Example processor(s) 220 include, but are not limited to, CPUs, Graphics Processing Units (GPUs), digital signal processors (DSPs), application specific integrated circuits (ASICs).

Turning back to FIG. 1, the difference in time it takes for the pressure wave 122 to arrive at the first 112 and second 114 detectors is indicative of the velocity of blood flow in the vessel 118. Accordingly, the processor 220 is configured to determine a first time at which the pressure wave is detected by the first detector and a second time at which the pressure wave is detected by the second detector and calculate a time difference between the first time and the second time. This time difference is then used by the processor to determine a velocity of the blood flowing in the blood vessel based on the time difference. Blood flow velocity may be determined based on a difference in the time it takes the propagating pressure wave to reach each detector, a difference in phase of the pressure wave detected at each detector, or a difference in frequency of the pressure wave detected at each detector.

Further, information regarding blood pressure may be inferred from the blood flow velocity detected by the device. For example, the device 100 may be used to determine relative changes in blood pressure. Calibration of the device on a user-by-user basis can also be used to determine an absolute measurement of blood pressure. The device 100 may also be used to measure blood flow at various points along a vein or other vessel. Changes in blood flow can be used to identify blockages in the vasculature. Properties, such as elasticity, of vessel walls may also be inferred from the transit time of the pressure wave, which can be indicative of vascular health.

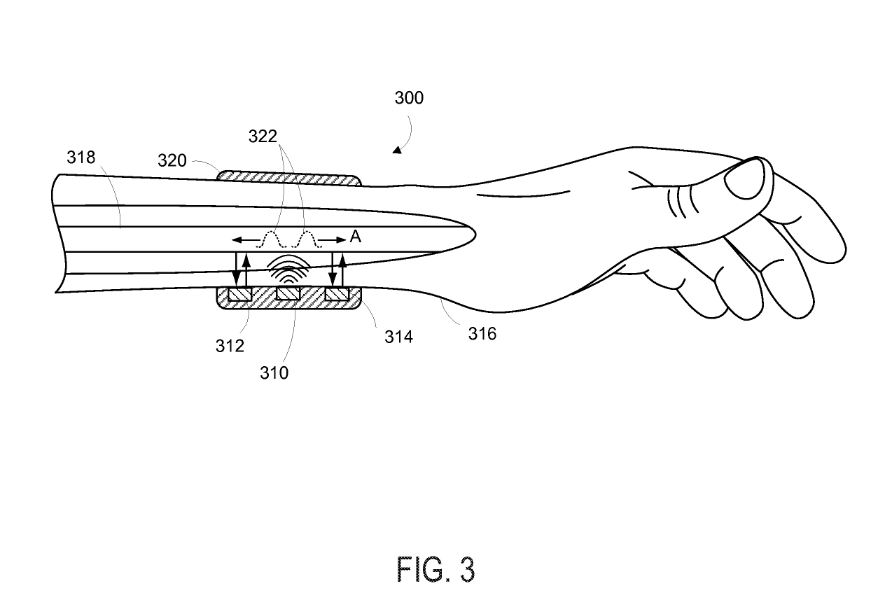

FIG. 3 illustrates another embodiment of a device 300, which may be held against a portion of the body, such as a wrist 316, with a mount 320, such as a strap. In this example, the first detector 312 is positioned upstream of the wave source 310, with respect to the direction of blood flow A in the blood vessel 318. The second detector 314 is positioned downstream of the wave source 310, with respect to the direction of blood flow A. As described above with respect to the embodiment of FIG. 1, a pressure wave 322 induced by the wave source 310 will travel upstream and downstream away from the source, with respect to the flow of fluid in the vessel 318. In this embodiment, the flow of fluid in the vessel, in the direction A, will retard the rate at which the generated wave 322 reaches the first detector 312. Conversely, the flow of fluid in the vessel, in the direction A, will accelerate the rate at which the generated wave 322 reaches the second detector 314. Thus, the difference in time it takes the wave 322 to reach the first 312 and second detectors 314 will provide an indication of the blood flow velocity.

In some examples, the wave source and detectors of the devices 100, 300 may be provided as or integrated into a wearable device, such as a wrist-mounted device, an eye-mountable device, a head mountable device (HMD) or an orally-mountable device. The term “wearable device,” as used in this disclosure, refers to any device that is capable of being worn or mounted at, on, in or in proximity to a body surface, such as a wrist, ankle, waist, chest, ear, eye, head or other body part. As such, the wearable device can collect data while in contact with or proximate to the body. For example, the wearable device can be configured to be part of a contact lens, a wristwatch, a “head-mountable display” (HMD), an orally-mountable device such as a retainer or orthodontic braces, a headband, a pair of eyeglasses, jewelry (e.g., earrings, ring, bracelet), a head cover such as a hat or cap, a belt, an earpiece, other clothing (e.g., a scarf), and/or other devices. Further, the wearable device may be mounted directly to a portion of the body with an adhesive substrate, for example, in the form of a patch, or may be implanted in the body, such as in the skin or another organ.

While the embodiments illustrated in FIGS. 1 and 3 demonstrate the use of a device 100, 300 on a blood vessel, those of ordinary skill in the art will recognize that the device may be used to measure velocity of fluid flowing in other vessels, both inside and outside of the body.

Some embodiments of the devices 100, 300 may include privacy controls which may be automatically implemented or controlled by the wearer or user of the device. For example, where a wearer's collected data is uploaded to a cloud computing network for trend analysis by a clinician, the data may be treated in one or more ways before it is stored or used, so that personally identifiable information is removed. For example, a wearer's identity may be treated so that no personally identifiable information can be determined for the wearer, or a wearer's geographic location may be generalized where location information is obtained (such as to a city, ZIP code, or state level), so that a particular location of a wearer cannot be determined.

Additionally or alternatively, wearers or users of a device may be provided with an opportunity to control whether or how the device collects information about the wearer (e.g., information about a wearer's medical history, social actions or activities, profession, a wearer's preferences, or a wearer's current location), or to control how such information may be used. Thus, the wearer may have control over how information is collected about him or her and used by a clinician or physician or other wearer of the data. For example, a wearer may elect that data collected from his or her device may only be shared with certain parties or used in certain ways.

III. Example Methods

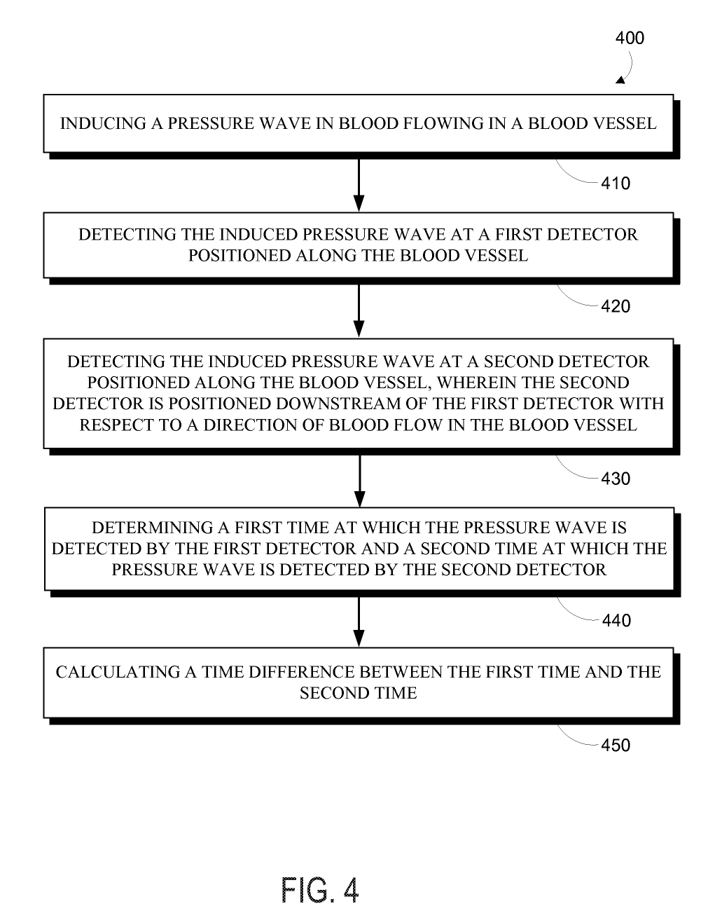

FIG. 4 is a flowchart of a method 400 for measuring blood flow velocity. Any suitable device, including devices 100, 300 described above, may be used to carry out the steps of the method 400. In a first step, a pressure wave is induced in blood flowing in a blood vessel. (410). As described above, the pressure wave may, for example, be acoustically, mechanically, electromagnetically induced. The induced pressure wave is detected at a first detector positioned along the blood vessel (420) and at a second detector positioned along the blood vessel (430). The second detector is positioned downstream of the first detector with respect to a direction of blood flow in the blood vessel. In some embodiments, the first detector is positioned upstream of the wave source, with respect to the direction of blood flow in the blood vessel. In other embodiments, both the first detector and the second detector are positioned downstream of the wave source, with respect to the direction of blood flow in the blood vessel.

A first time at which the pressure wave is detected by the first detector and a second time at which the pressure wave is detected by the second detector is determined (440) and a time difference between the first time and the second time is calculated (450), for example, by a processor. A velocity of the blood flowing in the blood vessel may be determined based on the calculated time difference.

The blood flow velocity measurements may be used to provide an indication of blood pressure. For example, blood flow velocity may also be determined over time, at a plurality of different times. A difference in velocity between each of the plurality of times may be determined and used to calculate a relative change in a blood pressure in the blood vessel.

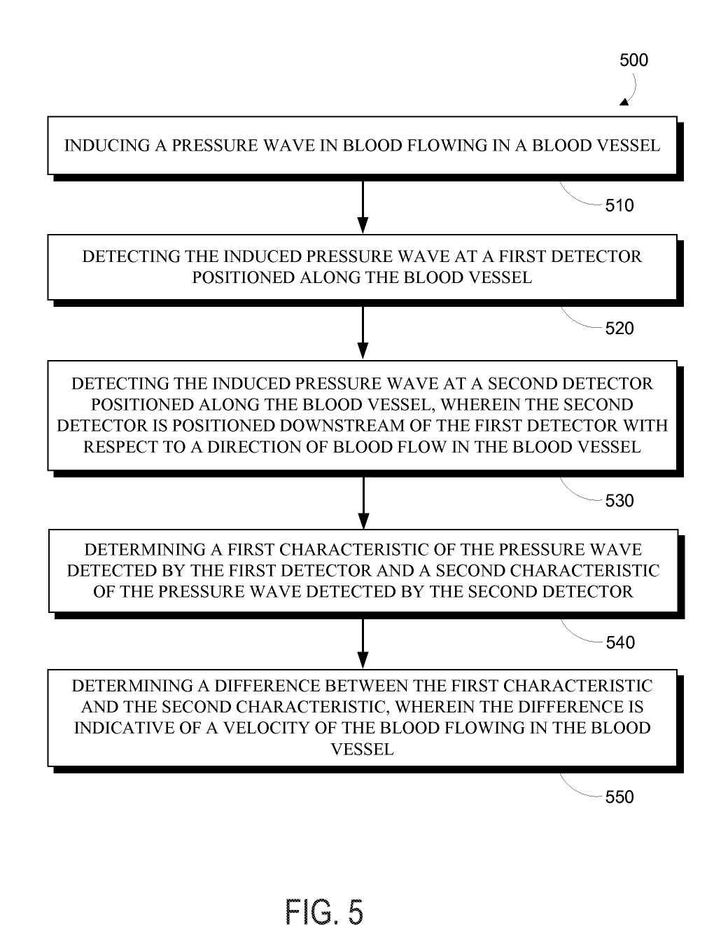

FIG. 5 is a flowchart of another method 500 for measuring blood flow velocity. Any suitable device, including devices 100, 300 described above, may be used to carry out the steps of the method 500. Similar to the method 400, in the method 500, a pressure wave is induced in blood flowing in a blood vessel (510), which is detected at a first detector (520) and a second detector (530), both positioned along the blood vessel. The second detector is positioned downstream of the first detector with respect to a direction of blood flow in the blood vessel. In some embodiments, the first detector is positioned upstream of the wave source, with respect to the direction of blood flow in the blood vessel. In other embodiments, both the first detector and the second detector are positioned downstream of the wave source, with respect to the direction of blood flow in the blood vessel.

A first characteristic of the pressure wave detected by the first detector and a second characteristic of the pressure wave detected by the second detector are determined. (540). A difference between the first characteristic and the second characteristic is also determined. (550). In one example, first characteristic is a time at which the pressure wave is detected by the first detector and the second characteristic is a time at which the pressure wave is detected by the second detector. In another example, the first characteristic is a phase of the pressure wave detected by the first detector and the second characteristic is a phase of the pressure wave detected by the second detector. The first and second characteristics may also be a frequency of the pressure wave detected by the first detector and the second detector, respectively. The difference in these detected characteristics (e.g., time, phase, frequency) is indicative of a velocity of the blood flowing in the blood vessel.

It will be readily understood that the aspects of the present disclosure, as generally described herein, and illustrated in the figures, can be arranged, substituted, combined, separated, and designed in a wide variety of different configurations, all of which are explicitly contemplated herein. While various aspects and embodiments have been disclosed herein, other aspects and embodiments will be apparent to those skilled in the art.

Example methods and systems are described above. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Reference is made herein to the accompanying figures, which form a part thereof. In the figures, similar symbols typically identify similar components, unless context dictates otherwise. Other embodiments may be utilized, and other changes may be made, without departing from the spirit or scope of the subject matter presented herein. The various aspects and embodiments disclosed herein are for purposes of illustration and are not intended to be limiting, with the true scope and spirit being indicated by the following claims.

What is claimed is:

1. A device, comprising:
a wave source configured to induce a pressure wave in blood flowing in a blood vessel;
a first detector positioned along the vessel and configured to detect a pressure wave in the blood flowing in the blood vessel; and
a second detector positioned along the vessel and configured to detect a pressure wave in the blood flowing in the blood vessel, wherein the second detector is positioned downstream of the first detector with respect to a direction of blood flow in the blood vessel.

2. The device of claim 1, wherein the wave source is an ultrasound transducer.

3. The device of claim 1, wherein the wave source is an electromagnetic source.

4. The device of claim 1, wherein the wave source is a mechanical source.

5. The device of claim 1, wherein the first detector is positioned upstream of the wave source, with respect to the direction of blood flow in the blood vessel.

6. The device of claim 1, wherein both the first detector and the second detector are positioned downstream of the wave source, with respect to the direction of blood flow in the blood vessel.

7. The device of claim 1, wherein both the first detector and second detector are ultrasound transducers.

8. The device of claim 1, further comprising:
a processor, wherein the processor is configured to:
determine a first time at which the pressure wave is detected by the first detector and a second time at which the pressure wave is detected by the second detector;
calculate a time difference between the first time and the second time;
determine a velocity of the blood flowing in the blood vessel based on the time difference.

9. A method, comprising:
inducing a pressure wave in blood flowing in a blood vessel;
detecting the induced pressure wave at a first detector positioned along the blood vessel;
detecting the induced pressure wave at a second detector positioned along the blood vessel, wherein the second detector is positioned downstream of the first detector with respect to a direction of blood flow in the blood vessel;
determining a first time at which the pressure wave is detected by the first detector and a second time at which the pressure wave is detected by the second detector; and
calculating a time difference between the first time and the second time.

10. The method of claim 9, further comprising:
determining a velocity of the blood flowing in the blood vessel based on the time difference.

11. The method of claim 9 further comprising:
determining a velocity of the blood flowing in the blood vessel at a plurality of times; and
calculating a relative change in a blood pressure in the blood vessel based on a difference in velocity determined between each of the plurality of times.

12. The method of claim 9, wherein the first detector is positioned upstream of the wave source, with respect to the direction of blood flow in the blood vessel.

13. The method of claim 9, wherein both the first detector and the second detector are positioned downstream of the wave source, with respect to the direction of blood flow in the blood vessel.

14. A method, comprising:
inducing a pressure wave in blood flowing in a blood vessel;
detecting the induced pressure wave at a first detector positioned along the blood vessel;
detecting the induced pressure wave at a second detector positioned along the blood vessel, wherein the second detector is positioned downstream of the first detector with respect to a direction of blood flow in the blood vessel;
determining a first characteristic of the pressure wave detected by the first detector and a second characteristic of the pressure wave detected by the second detector; and
determining a difference between the first characteristic and the second characteristic, wherein the difference is indicative of a velocity of the blood flowing in the blood vessel.

15. The method of claim 14, wherein the first characteristic is a time at which the pressure wave is detected by the first detector and the second characteristic is a time at which the pressure wave is detected by the second detector.

16. The method of claim 14, wherein the first characteristic is a phase of the pressure wave detected by the first detector and the second characteristic is a phase of the pressure wave detected by the second detector.

17. The method of claim 14, wherein the first characteristic is a frequency of the pressure wave detected by the first detector and the second characteristic is a frequency of the pressure wave detected by the second detector.

18. The device of claim 14, wherein the first detector is positioned upstream of the wave source, with respect to the direction of blood flow in the blood vessel.

19. The device of claim 14, wherein both the first detector and the second detector are positioned downstream of the wave source, with respect to the direction of blood flow in the blood vessel.

20. The method of claim 14 further comprising:
determining a velocity of the blood flowing in the blood vessel at a plurality of times; and
calculating a relative change in a blood pressure in the blood vessel based on a difference in velocity determined between each of the plurality of times.
